(19) United States
(12) Patent Application Publication
(10) Pub. No.: US 2026/0069708 A1
(43) Pub. Date: Mar. 12, 2026

(54) LISTERIA MONOCYTOGENES AS A VECTOR FOR TUMOR-SPECIFIC DELIVERY OF CHEMOTHERAPEUTIC AGENTS

(71) Applicants: TEXAS TECH UNIVERSITY SYSTEM, Lubbock, TX (US); THE TRUSTEES OF INDIANA UNIVERSITY, Bloomington, IN (US)

(72) Inventors: Laurence Wood, Lubbock, TX (US); Wyatt Paulishak, Lubbock, TX (US); Devin B. Lowe, Lubbock, TX (US); Michael VanNieuwenhze, Bloomington, IN (US); Jianen Lyu, Bloomington, IN (US)

(73) Assignees: TEXAS TECH UNIVERSITY SYSTEM, Lubbock, TX (US); THE TRUSTEES OF INDIANA UNIVERSITY, Bloomington, IN (US)

(21) Appl. No.: 19/110,069

(22) PCT Filed: Sep. 11, 2023
(86) PCT No.: PCT/US2023/032390

§ 371 Date: Mar. 8, 2025

Related U.S. Application Data
(60) Provisional application No. 63/405,315, filed on Sep. 9, 2022.

(51) Int. Cl. A61K 47/69; A61K 47/68; A61P 35/00

(52) U.S. Cl. CPC A61K 47/6901; A61K 47/68037; A61K 47/6809; A61K 47/6811; A61K 47/6889; A61P 35/00

(57) ABSTRACT

Embodiments of the present disclosure pertain to a modified Listeria monocytogenes bacterium (Listeria) that is associated with at least one therapeutic agent. The therapeutic agent may be associated with a surface of the Listeria through a Listeria binding agent. The therapeutic agent may also be covalently associated with the surface of the Listeria through a cleavable linker that directly links the therapeutic agent to the surface of the Listeria. Additional embodiments of the present disclosure pertain to methods of delivering at least one therapeutic agent to a subject by administering to the subject a Listeria of the present disclosure. Further embodiments of the present disclosure pertain to methods of making a Listeria of the present disclosure by associating the Listeria with at least one therapeutic agent.

### CROSS-REFERENCE TO RELATED APPLICATIONS

This application claims priority to U.S. Provisional Patent Application No. 63/405,315 , filed on Sep. 9, 2022. The entirety of the aforementioned application is incorporated herein by reference.

### BACKGROUND

Targeted delivery of drugs with antibody drug conjugates (ADCs) and liposome nanoparticles have numerous limitations, including poor cellular internalization and immune suppression. Numerous embodiments of the present disclosure aim to address the aforementioned limitations.

### SUMMARY

In some embodiments, the present disclosure pertains to a modified Listeria monocytogenes bacterium (Listeria) that is associated with at least one therapeutic agent. In some embodiments, the therapeutic agent is associated with a surface of the Listeria through a Listeria binding agent. In some embodiments, the Listeria binding agent is associated with the therapeutic agent and the surface of the Listeria. In some embodiments, the therapeutic agent is covalently associated with the Listeria binding agent through a cleavable linker.

In some embodiments, the therapeutic agent is covalently associated with the surface of the Listeria through a cleavable linker. In some embodiments, the cleavable linker directly links the therapeutic agent to the surface of the Listeria.

In some embodiments, the therapeutic agent includes, without limitation, an anti-cancer agent, a chemotherapeutic agent, non-radioactive compounds, cytotoxic proteins, derivatives thereof, and combinations thereof. In some embodiments, the therapeutic agent includes an anti-cancer agent. In some embodiments, the therapeutic agent includes a chemotherapeutic agent. In some embodiments, the chemotherapeutic agent includes, without limitation, Doxorubicin, Saporin, SN38, and combinations thereof.

In some embodiments, the Listeria is attenuated. In some embodiments, the attenuated Listeria includes a mutation or deletion in one or more virulence genes. In some embodiments, the one or more virulence genes include, without limitation, prfA, actA, hly, and combinations thereof.

Additional embodiments of the present disclosure pertain to methods of delivering at least one therapeutic agent to a subject by administering to the subject a Listeria of the present disclosure. In some embodiments, the method is utilized to treat or prevent a condition in the subject. In some embodiments, the condition is cancer, such as ovarian cancer, colorectal cancer, sarcoma, hepatocellular carcinoma, and combinations thereof.

Further embodiments of the present disclosure pertain to methods of making a Listeria of the present disclosure by associating the Listeria with at least one therapeutic agent. In some embodiments, the associating includes associating the therapeutic agent with the surface of the Listeria.

In some embodiments, the associating includes: covalently associating the therapeutic agent with a Listeria binding agent; and non-covalently associating the Listeria binding agent with the surface of the Listeria. In some embodiments, the associating includes covalently associating the therapeutic agent with the surface of the Listeria through a cleavable linker that directly links the therapeutic agent to the surface of the Listeria.

### DESCRIPTION OF THE DRAWINGS

FIGS. 1A and 1B provide illustrations of modified Listeria monocytogenes (LM) bacteria in accordance with the embodiments of the present disclosure.

FIG. 2 provides a hypothesized mechanism of LM delivery of antibody-drug conjugates (ADC) (A-H) and saporin (I-L) cargo into target cells. (A) LM binds either cMet or E-cadherin on cell surfaces triggering (B) endocytosis of the drug carrier. (C) LM is engulfed into an endosome which (D) recruits as lysosome for (E) fusion and formation of a degradative endolysosome. (F) LM and ADC are degraded resulting in release of the SN38/Dox cargo which diffuse into the nucleus of target cells resulting in (G) impaired topoisomerase I activity or (H) intercalates into DNA disrupting replication. In an alternative approach, (I) LM engulfment and degradation are disrupted by endosome degradation resulting in (J) LM escape into cell cytoplasm. (K) Cytoplasmic glutathione (GSH) releases surface bound saporin cargo. (L) Free saporin catalyzes the deactivation of cytoplasmic ribosomes resulting in disrupted translation. (M). Both ADC and saporin methods of LM drug delivery result in target cell death.

FIGS. 3A-3C provide data related to the characterization of various LM strains. FIG. 3A compares the infectivity of attenuated LM strain LM LLO-Ova (attenuated vaccine strain), ΔactA strain 4029, Δhly (LLO), and WT 10403S in J774 cell lines, as evaluated by a colony forming unit (CFU) counting infectivity assay. Relative infectivity was determined by evaluating total CFU numbers at increasing multiplicity of infection (MOIs) and at what MOI intracellular LM became susceptible to gentamycin treatment. Cells were infected for 3 hr before being washed and treated for 1 hr with 50 ug/mL gentamycin. FIG. 3B provides data where infectivity was secondarily evaluated using a fluorescent immunoassay in which total fluorescence of infected cells in a 96-well plate was measured. Cells were infected for 3 hr before being washed, treated with gentamycin, and fixed with 4% paraformaldehyde. FIG. 3C shows representative images of infected cells from an immunoassay, which were taken with nuclei stained using DAPI and LM stained using a polyclonal anti-LM Ab following permeabilization of the cells. Images were enhanced using ImageJ to show localization of LM fluorescent staining to cells.

FIGS. 4A-4C show data related to the cytotoxicity of LM strains. FIG. 4A compares the cytotoxicity of attenuated LM strain LM LLO-Ova, 4029, Δhly, and 10403S in J774 cell line at increasing MOI, as determined by a sulforhodamine B (SRB) assay. Cells were infected for 3 hr before being washed and incubated for 48 hr with 5 ug/mL gentamycin. FIG. 4B provides data where the cytotoxicity of LM strains was secondarily evaluated using flow cytometry for Annexin V (apoptosis) and PI (necrosis) with percent of total population illustrated. Cells were infected for 3 hr with 100:1 MOI LM before being washed and incubated for another 3 hr with 5 ug/mL gentamycin. Following infection and incubation period, cells were harvested and stained for analysis. FIG. 4C shows flow cytometry gating windows following LM infection and staining, illustrating the impact on apoptotic and necrotic cell populations following LM infection.

FIGS. 5A-5B show the Impact of PBS incubation and polyclonal anti-LM labeling with LM LLO-Ova on infectivity (FIG. 5A) and cytotoxicity (FIG. 5B) of J774 cells.

FIG. 5C shows the efficacy of SN38/Dox delivery by LM LLO-Ova in J774, as measured by an SRB cell viability assay. ADC attachment was performed by resuspending LM in PBS with anti-LM Ab/ADC and incubated for 1 hr at RT with periodic mixing.

FIGS. 6A-6D show the characterization of various LLM strains. FIG. 6A shows the phase contrast and fluorescence images of LM LLO-Ova cells stained with GFP without GSH treatment. FIG. 6B shows phase contrast and fluorescence images of LM LLO-Ova cells stained with sfGFP and then treated with 100 mM GSH at 37° C. for 30 min. FIG. 6C shows a number of sfGFP molecules released by each LM LLO-Ova cell and fluorescence intensity of bacterial cells after treatment with PBS or GSH at 37° C. for 30 min. FIG. 6D shows a phase contrast and fluorescence images of J774 cells infected with sfGFP labeled LM LLO-Ova. Scale bars represent 10 μm unless otherwise noted.

FIGS. 7A-7D show the cytotoxicity curves of various cell lines treated with LLM strains. FIG. 7A shows the cytotoxicity curve of J774 cell lines exposed to LM LLO-Ova cells treated with linker A only, modified saporin, unmodified saporin and untreated cells at different MOIS. FIG. 7B shows the cytotoxicity curve of J774 cell lines exposed to LM LLO-Ova cells treated with linker A only, modified saporin, and unmodified saporin at a narrower range of MOIS. FIG. 7C shows the cytotoxicity curve of J774 cell lines exposed to LM-saporin at different labeling concentrations at 500 MOI. FIG. 7D shows the cytotoxicity curve of J774 cell lines exposed to LM-saporin at different labeling concentrations.

FIGS. 8A-8F provide various schemes for the use of linkers A and B. FIG. 8A shows the structure of linker A and a scheme of synthesis for linker B. FIG. 8B shows the synthesis scheme for linking modified saporin to linker B. FIG. 8C provides a scheme for linking LM to linker A and labeling the linker with sfGFP. FIG. 8D provides a scheme for release of sfGFP. FIG. 8E provides a scheme for linking LM to linker A and coupling linker A to modified saporin. FIG. 8F provides a scheme for release of modified saporin.

FIGS. 9A-9C provide various schemes for the use of doxorubicin. FIG. 9A provides a synthetic scheme for modified doxorubicin. FIG. 9B provides a labeling scheme for doxorubicin. FIG. 9C provides a scheme for release of doxorubicin.

FIGS. 10A-10B illustrate differences in infectivity for LM LLO-Ova in additional cancer cell lines. FIG. 10A illustrates a comparison of infectivity between murine colorectal cancer cell lines CT26 and MC38 with J774. FIG. 10B illustrates comparison of infectivity between murine ovarian cancer ID8 cell line and human ovarian cancer cell line OVCAR-5.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Cancer chemotherapy frequently relies on systemic treatment with cytotoxic agents targeting critical cell pathways. While systemic chemotherapy can be effective, it is commonly associated with severe side effects that can take a heavy physical and mental toll on patients. Frequently, the side effects associated with chemotherapy are associated with non-specific delivery and activity of a drug in non-cancerous tissues.

In an effort to prevent the issues of nonspecific chemotherapy delivery and to improve therapeutic efficacy, numerous drug vehicles have been developed, including antibody drug conjugates (ADCs) and nanoparticles. An unexplored area of chemotherapy, however, is the application of intracellular bacteria for the delivery of therapeutics.

ADCs rely on the high target affinity of antibodies to localize delivery of chemotherapeutics to tumor sites. A common strategy is targeting cell surface tumor specific antigens (TSA) or tumor associated antigens (TAA) preferentially expressed on the surface of cancer cells. TSAs, however, are rare and most ADCs rely upon internalization which TAAs frequently poorly facilitate. Furthermore, ADCs poorly penetrate tumors and are susceptible to the development of resistance in the event their surface target is downregulated or deleted in surviving cancer cells.

Liposome nanoparticles are the most commonly applied nanotechnology and rely on the enhanced permeability and retention (EPR) effect for uptake into tumors. Surface modification of liposomes with functional moieties can improve targeting and localization. An issue with liposome vehicles, however, is that they are immunosuppressive and, therefore, pro tumor growth, which conflicts with their role in cancer therapy.

Therefore, despite decades of advances in chemotherapy and targeted delivery, there is still a need for novel technologies that improve drug delivery to tumors. Live bacteria cancer treatment predates chemotherapy and originates with the streptococcal bacteria treatments of sarcoma. Streptococcal treatments in patients resulted in moderate success but were largely abandoned for decades in favor of chemotherapeutics and radiation therapy approaches, which did not rely on living, infectious, and hazardous bacterial agents.

In the modern day, bacteria in cancer therapy has seen a resurgence because of their natural tumor tropism and the development of improved methods of studying cancer treatment response. Listeria monocytogenes (LM) is a Gram-positive intracellular bacterium that is commonly utilized as an anticancer vaccine platform.

Attenuated LM strains have been developed that improve the safety of LM therapies while still maintaining their anti-cancer properties. Over the last decades, LM has additionally been demonstrated as a delivery vehicle for radiotherapy, nanoparticle-bound DNA, and genome incorporating cDNA.

The intracellular life cycle of LM, during which LM gains access to both endosomal and cytoplasmic spaces, significantly contributes to LM's versatility as an anticancer platform. LM can infect antigen presenting cells and cancer cells, thereby resulting in beneficial immune activity against primary and metastatic tumors.

However, LM remains unexplored as a chemotherapy delivery vehicle. Additionally, LM drug delivery systems face numerous challenges, including sufficient invasion by payload-bearing Listeria, the release of cytotoxic payload, and delivery of drugs at sufficient intracellular concentration for cytotoxicity in target cancer cells. Numerous embodiments of the present disclosure aim to address the aforementioned limitations.

### Modified Listeria monocytogenes Bacteria

In some embodiments, the present disclosure pertains to a modified Listeria monocytogenes bacterium (Listeria) that is associated with at least one therapeutic agent. In some embodiments, the Listeria of the present disclosure represent an intracellular delivery vehicle. As set forth in more detail herein, the Listeria of the present disclosure can have numerous embodiments.

### Therapeutic Agents

The Listeria of the present disclosure can be associated with various therapeutic agents. For instance, in some embodiments, the therapeutic agent associated with the Listeria includes, without limitation, an anti-cancer agent, a chemotherapeutic agent, non-radioactive compounds, cytotoxic proteins, derivatives thereof, and combinations thereof. In some embodiments, the therapeutic agent lacks radioactive compounds. In some embodiments, the therapeutic agent includes an anti-cancer agent. In some embodiments, the therapeutic agent includes a chemotherapeutic agent. In some embodiments, the chemotherapeutic agent includes, without limitation, Doxorubicin, Saporin, SN38, and combinations thereof.

In some embodiments, the therapeutic agent includes Doxorubicin, SN38, derivatives thereof, or combinations thereof. In some embodiments, the therapeutic agent includes Saporin, Doxorubicin, derivatives thereof, or combinations thereof.

### Listeria

The Listeria of the present disclosure may be in various forms. For instance, in some embodiments, the Listeria is attenuated. In some embodiments, the attenuated Listeria includes a mutation or deletion in one or more virulence genes. In some embodiments, the one or more virulence genes include, without limitation, prfA, actA, hly, and combinations thereof. In some embodiments, the attenuated Listeria contains an anti-cancer vaccine plasmid. In some embodiments, the anti-cancer vaccine plasmid encodes an anti-cancer vaccine antigen conjugated to a truncated Listeriolysin (LLO) protein.

In some embodiments, the Listeria is suitable for use in delivering the therapeutic agent to a cell. In some embodiments, the Listeria is suitable for use in delivering the therapeutic agent to a subject. In some embodiments, the Listeria is suitable for use in treating or preventing a condition in a subject. In some embodiments, the Listeria is suitable for use in treating or preventing a cancer in a subject. In some embodiments, the Listeria is suitable for use in treating or preventing ovarian cancer, colorectal cancer, sarcoma, and/or hepatocellular carcinoma in a subject.

Association of therapeutic agents with Listeria

The Listeria of the present disclosure can be associated with one or more therapeutic agents in various manners. For instance, in some embodiments, the therapeutic agent is non-covalently associated with the Listeria. In some embodiments, the therapeutic agent is covalently associated with the Listeria. In some embodiments, the therapeutic agent is associated with the cytoplasm of the Listeria. In some embodiments, the therapeutic agent is associated with the surface of the Listeria.

In some embodiments, the therapeutic agent is directly associated with the surface of the Listeria. In some embodiments, the therapeutic agent is directly associated with the surface of the Listeria through a covalent bond. In some embodiments, the therapeutic agent is covalently associated with the surface of the Listeria through a cleavable linker. In some embodiments, the therapeutic agent is covalently associated with the surface of the Listeria through a non-cleavable linker.

In some embodiments, the therapeutic agent is indirectly associated with the surface of the Listeria. In some embodiments, the therapeutic agent is associated with the surface of the Listeria through a Listeria binding agent. In some embodiments, the Listeria binding agent is associated with the therapeutic agent and the surface of the Listeria. In some embodiments, the Listeria binding agent is covalently associated with the therapeutic agent and non-covalently associated with the surface of the Listeria. In some embodiments, a first end of the Listeria binding agent is associated with the therapeutic agent and a second end of the Listeria binding agent is associated with the surface of the Listeria.

In some embodiments, the therapeutic agent is covalently associated with the Listeria binding agent. In some embodiments, the therapeutic agent is covalently associated with the Listeria binding agent through a linker. In some embodiments, the therapeutic agent is covalently associated with the Listeria binding agent through a cleavable linker. In some embodiments, the therapeutic agent is covalently associated with the Listeria binding agent through a non-cleavable linker. In some embodiments, the Listeria binding agent may include additional functional moieties to enable effective attachment of a therapeutic agent onto the Listeria binding agent. In some embodiments, such moieties can include, without limitation, esters, non-cleavable linkers, chemical binding moieties that covalently modify a Listeria surface, and combinations thereof.

In some embodiments, the Listeria includes a plurality of different therapeutic agents that are associated with a plurality of Listeria binding agents. In some embodiments, the Listeria includes a plurality of different therapeutic agents and a plurality of different Listeria binding agents. In some embodiments, the different therapeutic agents are associated with different Listeria binding agents. In some embodiments, the Listeria includes at least a first therapeutic agent associated with a first Listeria binding agent, and a second therapeutic agent associated with a second Listeria binding agent.

The Listeria of the present disclosure can be associated with various Listeria binding agents. For instance, in some embodiments, the Listeria binding agent includes an antibody or a fragment thereof. In some embodiments, the Listeria binding agent includes an anti-Listeria antibody.

Therapeutic agents may be associated with Listeria binding agents at various ratios. For instance, in some embodiments, the therapeutic agent to Listeria binding agent ratio (i.e., the ratio of the number of therapeutic agents per Listeria binding agent) is at least 1. In some embodiments, the therapeutic agent to Listeria binding agent ratio is at least 2. In some embodiments, the therapeutic agent to Listeria binding agent ratio is at least 3. In some embodiments, the therapeutic agent to Listeria binding agent ratio is at least 4. In some embodiments, the therapeutic agent to Listeria binding agent ratio is at least 5. In some embodiments, the therapeutic agent to Listeria binding agent ratio is at least 6. In some embodiments, the therapeutic agent to Listeria binding agent ratio is at least 7.

In some embodiments, the therapeutic agent is covalently associated with the surface of the Listeria through a cleavable linker. In some embodiments, the cleavable linker directly links the therapeutic agent to the surface of the Listeria.

In some embodiments, the therapeutic agent is covalently associated with the surface of the Listeria through a non-cleavable linker. In some embodiments, the non-cleavable linker directly links the therapeutic agent to the surface of the Listeria.

Therapeutic agents may be associated with a surface of a Listeria or a Listeria binding agent through various types of linkers. For instance, in some embodiments, the linker includes a non-cleavable linker. In some embodiments, the linker includes a cleavable linker. In some embodiments, the cleavable linker includes an enzyme cleavable linker. In some embodiments, the enzyme cleavable linker is an esterase cleavable linker. In some embodiments, the cleavable linker is cleavable in the presence of one or more reducing agents. In some embodiments, the cleavable linker is cleavable in the presence of one or more reducing agents in an intracellular microenvironment. In some embodiments, the cleavable linker includes a disulfide bond.

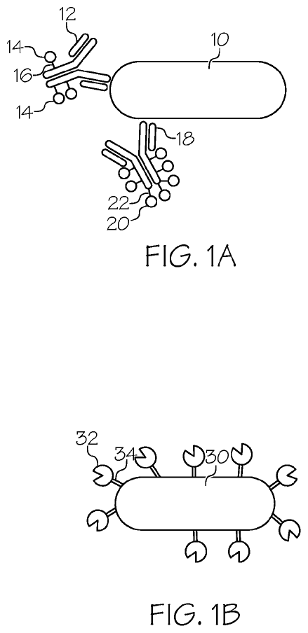

The Listeria of the present disclosure can have numerous embodiments. For instance, a specific example of a Listeria of the present disclosure is illustrated in FIG. 1A as Listeria 10. In this Example, the surface of Listeria 10 is associated with a first therapeutic agent 14 and a second therapeutic agent 20. First therapeutic agent 14 is associated with the surface of Listeria 10 through Listeria binding agent 12, which is simultaneously associated with therapeutic agent 14 through a cleavable linker 16, and the surface of Listeria 10 through non-covalent binding. Similarly, second therapeutic agent 20 is associated with the surface of Listeria 10 through Listeria binding agent 18, which is simultaneously associated with therapeutic agent 20 through a cleavable linker 22, and the surface of Listeria 10 through non-covalent binding. In a more specific embodiment, therapeutic agents 14 and 20 may include Doxorubicin and SN38, respectively. In such embodiments, Listeria 10 may be suitable for use in treating a cancer, such as ovarian cancer, colorectal cancer, sarcoma, and/or hepatocellular carcinoma.

Another specific example of a Listeria of the present disclosure is illustrated in FIG. 1B as Listeria 30. In this Example, the surface of Listeria 30 is associated with therapeutic agent 32 through a linker 34, which directly links therapeutic agent 32 to the surface of Listeria 30. In a more specific embodiment, therapeutic agent 32 may include Saporin, Doxorubicin, derivatives thereof, or combinations thereof.

### Methods of Delivering Therapeutic Agents to Cells

Additional embodiments of the present disclosure pertain to methods of delivering a therapeutic agent to a cell. In some embodiments, the methods of the present disclosure include associating the cell with a modified Listeria monocytogenes bacterium (Listeria) of the present disclosure, which is associated with the therapeutic agent.

In some embodiments, the associating includes incubating the cell with the Listeria. In some embodiments, the associating occurs in vitro. In some embodiments, the associating occurs in vivo in a subject.

In some embodiments, the cell includes a tumor cell. In some embodiments, the tumor cell includes an ovarian cancer cell line, a colorectal cancer cell line, a sarcoma cell line, a hepatocellular carcinoma cell line, or combinations thereof.

### Methods of Delivering Therapeutic Agents to Subjects

Further embodiments of the present disclosure pertain to methods of delivering at least one therapeutic agent to a subject. In some embodiments, such methods include administering to the subject a modified Listeria monocytogenes bacterium (Listeria) of the present disclosure, which is associated with the therapeutic agent.

Suitable Listeria were described supra and are incorporated herein by reference. Various methods may be utilized to administer the Listeria of the present disclosure to a subject. For instance, in some embodiments, the administering occurs by a method that includes, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, systemic administration, local administration, and combinations thereof.

In some embodiments, the administration includes systemic administration to a subject. In some embodiments, the administration includes local administration to a specific tissue of a subject. In some embodiments, the tissue includes a tumor.

The Listeria of the present disclosure may be administered to various subjects. For instance, in some embodiments, the subject is a human being. In some embodiments, the subject is suffering from a cancer. In some embodiments, the subject is vulnerable to a cancer.

In some embodiments, the methods of the present disclosure may be utilized to treat or prevent a condition in a subject. In some embodiments, the condition is cancer. In some embodiments, the cancer is ovarian cancer, colorectal cancer, hepatocellular carcinoma, sarcoma, and combinations thereof. In some embodiments, the cancer is ovarian cancer. In some embodiments, the cancer is sarcoma.

In some embodiments, the Listeria is administered to the subject in combination with one or more additional treatments. In some embodiments, the one or more additional treatments include, without limitation, anti-cancer vaccines, chemotherapy, radiotherapy, immunotherapy, or combinations thereof.

Without being bound by theory, the methods of the present disclosure may deliver one or more therapeutic agents to a subject through various mechanisms. For instance, in some embodiments, the administered Listeria enters the cells of a subject through endocytosis. Thereafter, therapeutic agents associated with the Listeria are released. For instance, in some embodiments where the therapeutic agent is associated with the Listeria through a cleavable linker, the therapeutic agent is released after cleavage of the cleavable linker. In some embodiments, the cleavable linker is cleaved in response to the intracellular reducing environment. Thereafter, the released therapeutic agent may initiate a cellular effect, such as apoptosis.

### Methods of Making Modified Listeria monocytogenes Bacteria

Further embodiments of the present disclosure pertain to methods of making a modified Listeria monocytogenes bacterium (Listeria). Such methods generally include associating the Listeria with at least one therapeutic agent.

Therapeutic agents may be associated with Listeria in various manners. For instance, in some embodiments, the associating includes associating the therapeutic agent with the surface of the Listeria. In some embodiments, the associating includes: associating the therapeutic agent with a Listeria binding agent; and associating the Listeria binding agent with the surface of the Listeria. In some embodiments, the associating includes: covalently associating the therapeutic agent with a Listeria binding agent; and covalently associating the Listeria binding agent with the surface of the Listeria.

In some embodiments, the Listeria binding agent includes an antibody or a fragment thereof. In some embodiments, the associating of the therapeutic agent with the Listeria binding agent includes covalently associating the therapeutic agent with the Listeria binding agent through a cleavable linker. In some embodiments, the associating of the therapeutic agent with the Listeria binding agent includes covalently associating the therapeutic agent with the Listeria binding agent through a non-cleavable linker. In some embodiments, the associating of the therapeutic agent with the Listeria binding agent includes associating a plurality of different therapeutic agents with a plurality of Listeria binding agents. In some embodiments, the associating of the therapeutic agent with the Listeria binding agent includes associating a first therapeutic agent with a first Listeria binding agent and associating a second therapeutic agent with a second Listeria binding agent.

In some embodiments, a therapeutic agent may be associated with a Listeria directly. For instance, in some embodiments, the associating includes covalently associating the therapeutic agent with the surface of the Listeria through a cleavable linker. In some embodiments, the associating includes covalently associating the therapeutic agent with the surface of the Listeria through a non-cleavable linker. In some embodiments, the linker (e.g., cleavable linker) directly links the therapeutic agent to the surface of the Listeria.

The methods of the present disclosure may utilize various linkers. Suitable linkers were described supra and are incorporated herein by reference. For instance, in some embodiments, the linker includes a non-cleavable linker. In some embodiments, the linker includes a cleavable linker. In some embodiments, the cleavable linker includes an enzyme cleavable linker. In some embodiments, the enzyme cleavable linker is an esterase cleavable linker. In some embodiments, the cleavable linker is cleavable in the presence of one or more reducing agents. In some embodiments, the cleavable linker is cleavable in the presence of one or more reducing agents in an intracellular microenvironment. In some embodiments, the cleavable linker includes a disulfide bond.

The methods of the present disclosure may associate various therapeutic agents with Listeria. Suitable therapeutic agents were also described supra and are incorporated herein by reference. For instance, in some embodiments, the therapeutic agents include, without limitation, an anti-cancer agent, a chemotherapeutic agent, non-radioactive compounds, cytotoxic proteins, Doxorubicin, Saporin, SN38, derivatives thereof, and combinations thereof.

The methods of the present disclosure may also associate therapeutic agents with various types of Listeria. Suitable Listeria were also described supra and are incorporated herein by reference. For instance, in some embodiments, the Listeria is attenuated. In some embodiments, the attenuated Listeria includes a mutation or deletion in one or more virulence genes. In some embodiments, the one or more virulence genes include, without limitation, prfA, actA, hly, and combinations thereof.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Listeria monocytogenes Mediated Delivery of ADC and Saporin Produces Payload Dependent Cell Death in J774 Sarcoma Cell Line

In this Example, Applicants demonstrate that Listeria monocytogenes (LM) labelled with noncovalent ADC and covalent surface payloads can deliver chemotherapeutic cargo and induce cytotoxicity J774 cells in vitro. Applicants'investigation of LM strains 10403S, 4029, LLO-Ova, and Ahly revealed that infectivity and cytotoxicity vary significantly between strain depending on degree of attenuation. Delivery of ADC cargo using the LM LLO-Ova vaccine strain was found to be effective but limited in efficacy while surface attachment of saporin cargo to LM improved cytotoxicity by approximately ten-fold.

Applicants' results demonstrate the viability of live LM as a chemotherapeutic delivery vehicle using both noncovalent and covalent payload loading mechanisms. Moreover, Applicants' results seek to leverage the invasiveness, immunogenicity, and inherent antitumor activity of LM to promote efficient drug delivery and enhance efficacy.

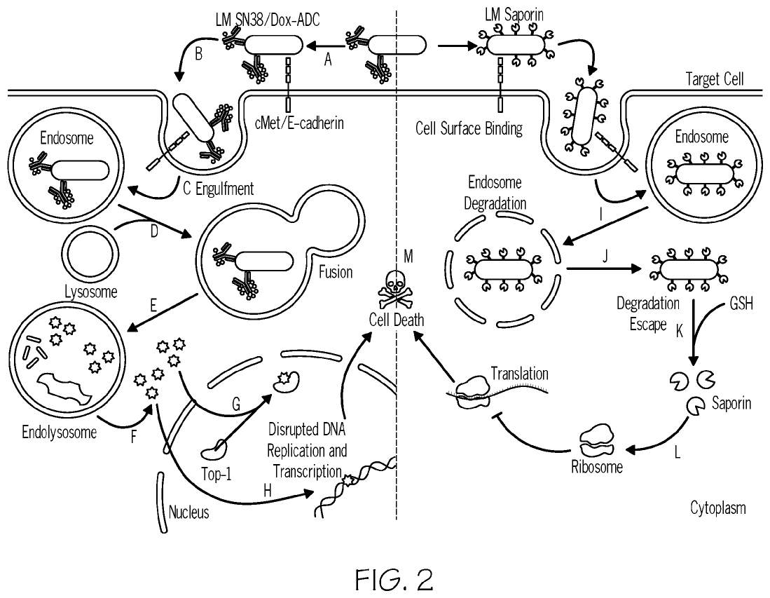

In this Example, Applicants developed two approaches of LM mediated chemotherapy delivery and release: one endolysosome targeting using SN38/Dox-ADCs and the other cytoplasm targeting using saporin (FIG. 2). Experimental validation of LM chemotherapy delivery was facilitated by two different labeling and release approaches in the J774 macrophage sarcoma-like cell line.

Applicants' first approach relies upon noncovalent binding of ADC payloads with the LM surface and release via endolysosome degradation comparing delivery of topoisomerase-1 (Top-1) inhibitor with SN38 and DNA replication inhibitor doxorubicin (Dox). SN38 is the active product of the clinically approved prodrug irinotecan. The SN38 and Dox-ADC payloads resulted in moderate but cell labeling dependent cell death, which was improved over the parent antibody (Ab).

Applicants' second approach utilized the ribosome inactivating saporin enzyme as payload. Chemical reduction and covalent attachment of saporin to the cell surface resulted in a high payload per LM and dramatically improved cytotoxicity over the first approach.

### Example 1.1. Infection Proficiency of LM Strains in J774 Cells

The infectivity of LM is expected to directly correlate with the amount of drug delivered and, therefore, proficiency as a drug delivery vehicle. Comparison of LM infectivity with strains differing in attenuation method is unreported in the literature. A gentamicin protection assay (GPA) was used to compare the infectivity of LM strains 10403S (WT), 4029 (ΔactA), Δhly, and LLO-Ova (pGG34 plasmid control vaccine strain). Both the 4029 and Δhly strains were originally developed from the WT LM with the former strain incapable of cell to cell spread and the later heavily attenuated in ability to escape endosome degradation. The LM LLO-Ova strain was developed from the XFL7 LM strain which itself was generated from the WT strain. XFL7 LM is attenuated by the deletion of the prfA gene, the master virulence regulator in LM, and was partially rescued to create the LLO-Ova strain using the pGG34 vaccine plasmid which encodes a mutated prfA gene.

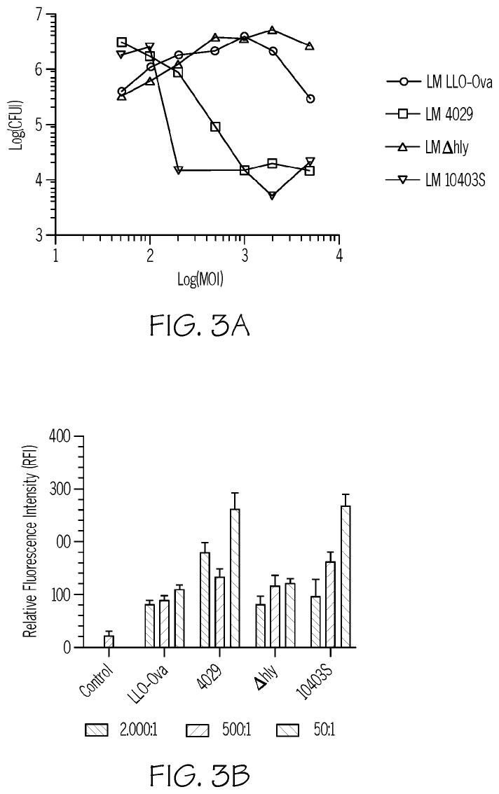

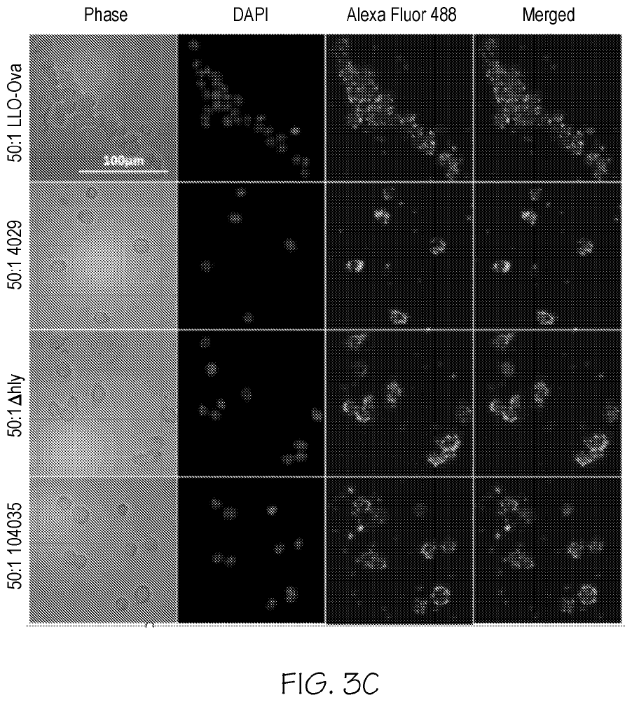

In the GPA, intracellular LM were counted following cell treatment with gentamicin, lysis, and titration on agar plates at increasing multiplicity of infection (MOI). LM strains WT and 4029 showed remarkably similar infectivity profiles in the J774 cell line (FIG. 3A). Similarly, the Δhly and LLO-Ova LM strains demonstrated very similar infection profiles. Unexpectedly, infection with strains Δhly and LLO-Ova resulted in a drop in colony forming units (CFU) at the highest MOIs while infection with WT and 4029 resulted in the same CFU drop but at lower MOI.

Initially, these results appeared to indicate that the LM Δhly and LLO-Ova strains are more infectious than the WT and 4029 strains. Furthermore, it was not discernable if the reduced CFU counts at increasing MOI in each strain was caused by gentamycin cell killing or if LM infectivity decreases at higher MOIs.

Higher LM infection is expected to cause an increase in cell membrane disruption. Applicants hypothesized that LM membrane disruption enables gentamicin to diffuse into infected cells and, at high MOI, achieve sufficient concentration to kill intracellular LM. To evaluate and determine whether the WT and 4029 LM are more infectious than the Δhly and LLO-Ova LM, Applicants utilized an immunofluorescence assay as a second method of measuring and comparing LM strain infectivity. Following permeabilization and immunofluorescence staining of LM, the infectivity again indicated a close similarity between the WT and 4029 strains along with the Δhly and LLO-Ova strains in J774 (FIG. 3B). In this analysis, the WT and 4029 strains resulted in higher fluorescence with an upward trend coinciding with increasing MOI. Therefore, Applicants confirmed that high MOIs of LM will disrupt the plasma membrane of infected cells and enable antibiotic diffusion and activity.

Fluorescence imaging at low MOI (50:1) also showed close association of LM with DAPI stained nuclei, indicating the immunostained bacteria were inside the cells (FIG. 3C). Increasing MOIs with each strain resulted in visibly increased diffusion of fluorescence in cells, confirming the total fluorescence measurements and results was due to increased cellular infection. These results indicate that the WT and 4029 strains are more infectious toward J774 cells than the Δhly and LLO-Ova strains.

### Example 1.2. Cytotoxicity of LM Strains in J774 Cells

Since LM is a toxic disease-causing pathogen, it was expected that infection of the J774 cell line will result in cytotoxicity correlating with the MOI during infection. Therefore, cytotoxicity of the four LM strains was evaluated to determine which strain may be a viable drug delivery vehicle. Ideally, the selected strain is cytotoxic to the cancer cells it is infecting as vehicle induced tumor lytic activity is beneficial to therapeutic efficacy.

To evaluate LM strain cytotoxicity, a GPA sulforhodamine B (SRB) cell viability assay was utilized. The SRB assay was chosen over other cytotoxic assays which measure metabolic activity to avoid inaccurate reading caused by LM metabolism.

For measurement of LM cytotoxicity, J774 cells were again infected at increasing MOIS but allowed to incubate after infection in the presence of low-concentration gentamicin. This low-concentration gentamicin prevents replication of LM that escape into the surrounding media.

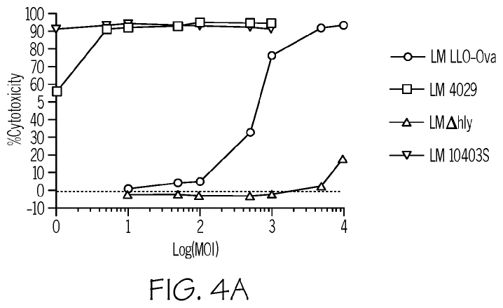

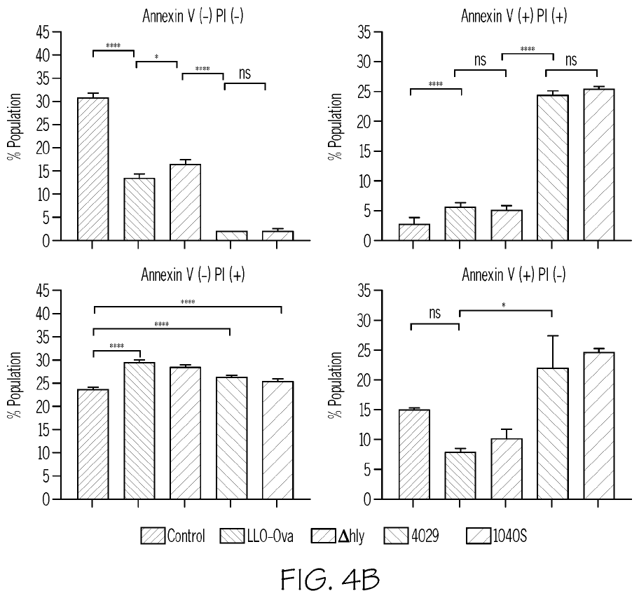

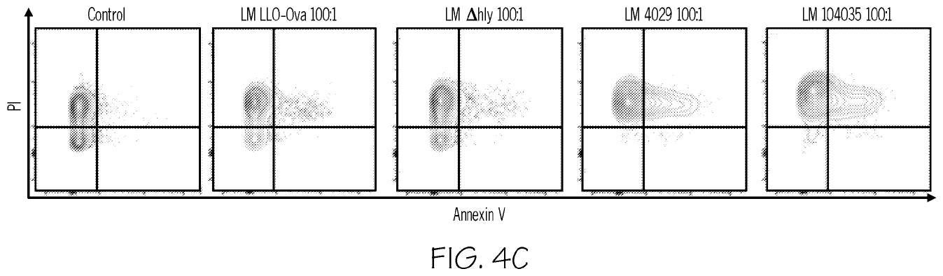

Higher infectivity is expected to result in higher cytotoxicity. As previous infectivity experiment indicated, both WT and 4029 strains had dramatically higher cytotoxicity than the LLO-Ova and Δhly strains (FIG. 4A). Interestingly, all four strains demonstrated differing cytotoxicity profiles in contrast to the paired infectivity profiles.

As a secondary method of evaluating cytotoxicity, the impact of LM infection on cell viability was measured by apoptotic annexin V signaling and necrosis propidium iodide (PI) staining using multicolored flow cytometry analysis. Infection with all strains resulted in a significant increase in the necrotic population of cells (FIGS. 4B and 4C). Again, infection with WT and 4029 strains paralleled each other resulting in a significant increase in the dual apoptotic and necrotic population at the 100:1 MOI.

Effect of infection on cell viability with LLO-Ova and Δhly strains was also in parallel but did not result in the same effect at the 100:1 MOI. Infection at 1,000:1 MOI with both WT and 4029 strains induced such stress that the infected cells burst during removal from the culture plate rendering them unable to be analyzed.

Increasing the MOI with the Δhly strain caused a further increase in the dual population. However, increasing the LM LLO-Ova MOI did not result in a further increase in the necrotic population.

### Example 1.3. Impact of LM Stock Preparation and Antibody Labeling on Infectivity and Cytotoxicity in J774

After evaluating both infectivity and cytotoxicity of the LM strains, it was determined that the LLO-Ova strain would be best suited to testing the ability of LM to deliver chemotherapeutic cargo. This decision was based on the LLO-Ova strain's medium cytotoxicity among the tested strains and a lower permeabilization of infected cells which could cause loss of delivered payload. Furthermore, the LM LLOOva strains are clinically relevant, and vehicle antitumor activity is expected to improve response.

With a LM strain selected for testing drug delivery, Applicants next evaluated the reproducibility of LM stock preparations and the impact of parent Ab labeling on the LM LLO-Ova strains infectivity and cytotoxicity. The consistency and effect of LM stock preparation methods on LM infectivity and cytotoxicity is unreported in literature. For LM to be viable as a drug delivery vehicle, stock to stock consistency is necessary for testing and dosing.

Since the J774 cell line is highly infected by LM, Applicants expected that differences between stocks would be more apparent and detectable. When the cytotoxicity of a one-day LM preparation method, which avoids time where LM is lek at stationary growth phase, was compared to the typical two-day LM preparation method, there was no statistical difference in cytotoxicity between either preparation method. This lack of statistical difference extended to variable duration of overnight incubation in the two-day method and there was no statistical difference in precision between the LM preparation methods.

Infectivity did not appear to be greatly affected by either preparation method. These results indicate that the standard method of LM preparation is consistent and robust.

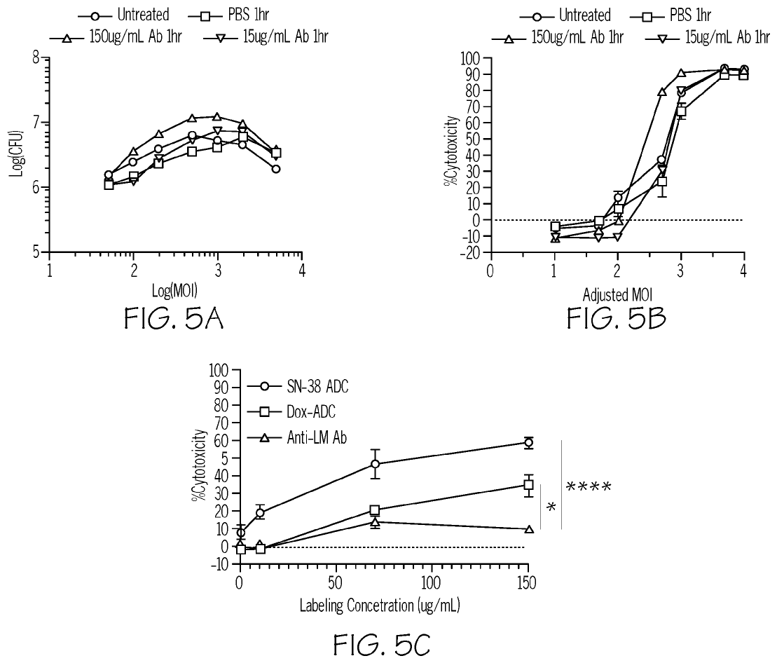

Applicants next evaluated the impact of Ab labeling of LM on the bacteria's ability to infect J774 cells. The surface proteins on LM that are recognized by the polyclonal Ab used in the synthesis of SN38/Dox-ADC are not reported and, therefore, could potentially block or modify LM J774 interaction and infection. When infectivity of untreated LM was compared to 1 hr incubation in PBS, there was a small drop in infectivity (FIG. 5A). This minor effect on infectivity was reversed when LM was labelled with increasing polyclonal Ab concentrations. These minor effects of Ab binding translated in cytotoxicity analysis where the only statistically significant effect was observed at the 500:1 MOI in the Ab labelled groups (FIG. 5B). From these evaluations of Ab labeling, Applicants concluded that the polyclonal Ab would not significantly impact evaluation of LM as a chemotherapeutic delivery vehicle.

### Example 1.4. LM Mediated Delivery of SN-38/Dox Anti-LM ADC

As an initial probe of LM drug delivery, Applicants next began development of ADC payloads suitable for delivery. For this purpose, Applicants synthesized a pair of anti-LM ADCs with SN38 and Dox payloads. SN38 is the active product of the prodrug irinotecan which, when activated intracellularly, blocks the activity of Top-1 resulting in impaired cell replication and death. The SN38 payload was chosen because literature reports indicate the chemotherapeutic has a poor permeability that can be improved through administration via various delivery mechanisms. Therefore, SN38 was deemed a good candidate for evaluating the ability of LM to deliver chemotherapeutic cargo since predominant intracellular delivery of SN38 will result in cell killing.

Dox was selected because it is among the most commonly reported drug payloads, is broadly usable in a wide range of cancers, and has been applied successfully with E. coli Nissle. Mechanistically, Dox is capable of diffusing into cells and intercalating into DNA, which disrupts replication and leads to cell death. To facilitate drug delivery, SN38-ADC drug antibody ratio (DAR) of ˜3 and Dox-ADC with a DAR of ˜7 was synthesized using a polyclonal anti-LM and ADC conjugation kits from CellMosaic. The SN38-ADC was synthesized with an ester linker while the Dox-ADC was synthesized with a non-cleavable linker. Ester linkers rely upon lysosomal esterase's for drug release while non-cleavable linker rely on total Ab degradation by the lysosome for release and are, therefore, slower to release payloads.

Experimental evaluation of LM mediated delivery of SN38 and Dox-ADC was performed using a GPA SRB cytotoxicity assay after infection of J774 with labelled bacteria. Extracellular ADC is washed away following LM infection and, therefore, cytotoxicity is limited to delivery by LM. Infection with LM labelled at increasing concentrations of SN38-ADC resulted in a labelling concentration dependent increase in cytotoxicity (FIG. 5C). This cytotoxicity appeared to plateau at ˜60% and was significantly higher than the LM labelled with the parent Ab at the same concentration. This level of cell killing was not matched by the Dox-ADC but was still significantly higher than the parent Ab. Critically, this cytotoxicity was produced after only a three-hour infection period, after which, no additional SN38, Dox, or LM was administered to cells and any extracellular payload is washed away.

When LM delivery was repeated with labeling at a higher LM density, there was no cytotoxicity in the J774 cell line (data not shown). These results indicated that LM mediated delivery of cytotoxic payloads is viable but is dependent on payload-per-LM for efficacy. Furthermore, release condition appears to be an important factor in producing cytotoxicity.

Dox reportedly has a 10-100 fold lower IC₅₀ than irinotecan while the Dox-ADC was less cytotoxic than the SN38-ADC despite a higher DAR. It is plausible that the non-cleavable linker in the Dox-ADC significantly reduced the rate of drug release inside target cells and thereby reduces cytotoxicity.

These results, coupled with the difficulty of scaling ADC production to sufficiently label LM for in vivo application, elicited Applicants to begin investigating covalent attachment to the LM surface as a means of improving cytotoxicity. Initially, a Dox conjugate was designed for covalent attachment to the surface of LM. However, it was found that the Dox payload would precipitate from solution during LM labeling, thereby severely limiting delivery efficiency. Therefore, Applicants began investigating the application of more potent enzymatic payloads for delivery by LM.

### Example 1.5. LM Surface Labeling with sfGFP and Saporin

Protein toxins are highly potent and cytotoxic because they are highly specific and efficient inducers of cytotoxicity. Ricin, for example, is a notorious plant toxin that has an LD₅₀ of 3-5μg/kg. Ricin is classified as a type II ribosome-inactivating protein (RIP) and contains two domains: ricin toxin A (RTA) chain and ricin toxin B (RTB) chain. The RTA chain is the catalytic enzyme domain that irreversibly deactivates eukaryotic ribosomes by cleaving an essential adenine residue in the 28S rRNA active site of the 60S subunit.

Enzymatic cleavage inhibits protein production and eventually leads to apoptosis. The RTB chain facilitates cell invasion via binding to terminal galactose residues on cell membrane glycoproteins thereby triggering endocytosis. RIPs such as ricin, are considered highly efficient such that it is hypothesized one RIP molecule is sufficient to cause cell death and theoretically corresponds to sub-picomolar intracellular concentration (assuming a cell diameter of 20 μm).

Purified RTA has been reportedly conjugated to antibodies to selectively kill target cancer cells. However, the high toxicity of ricin makes it dangerous to extract and purify RTA, so the protein saporin is used more frequently as a replacement for RTA. Unlike ricin, saporin is a type I RIP and only contains the enzymatic moiety that functions like RTA.

Consequently, saporin does not have an efficient cell entry pathway and thus is a thousandfold less toxic than ricin. The difference in intracellular and extracellular toxicity made saporin a suitable candidate as the payload for delivery by LM LLO-Ova. To facilitate verification of protein payload loading on LM, superfolder green fluorescent protein (sfGFP) was used as a model protein to enable visualization of labeling, quantification, and intracellular delivery.

Cells labeled with GFP and saporin involve two bifunctional linkers, linker A and linker B. Linker B was used for modification of saporin via covalent attachment to its N-terminal amine group. Protein labeling of LM LLO-Ova begins with installation of linker A onto the N-terminal amine group of bacterial surface proteins, followed by treatment of sfGFP or modified saporin. The payload can be released via cleavage of the disulfide bond upon exposure to reducing intracellular microenvironment of target cells. The released saporin subsequently initiates a self-immolative reaction, yielding free saporin in its unmodified form (FIGS. 8A-8F).

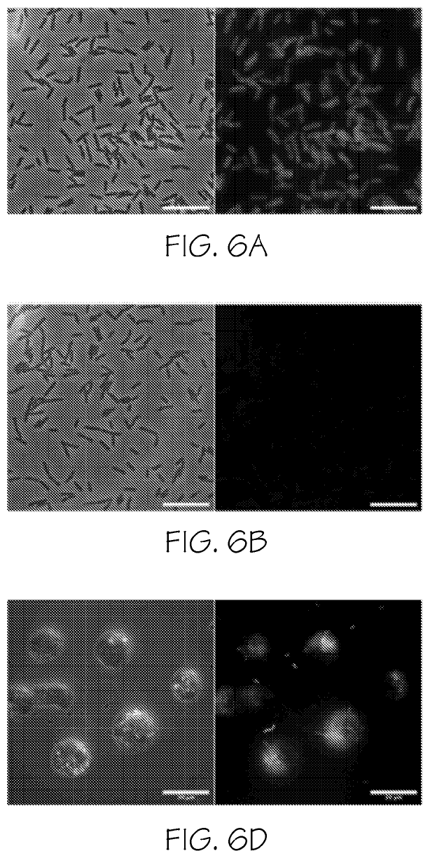

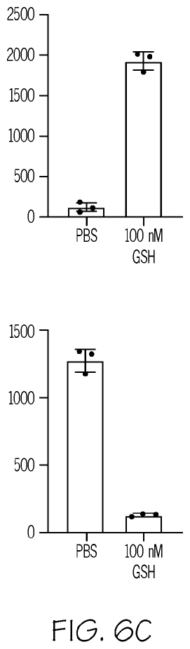

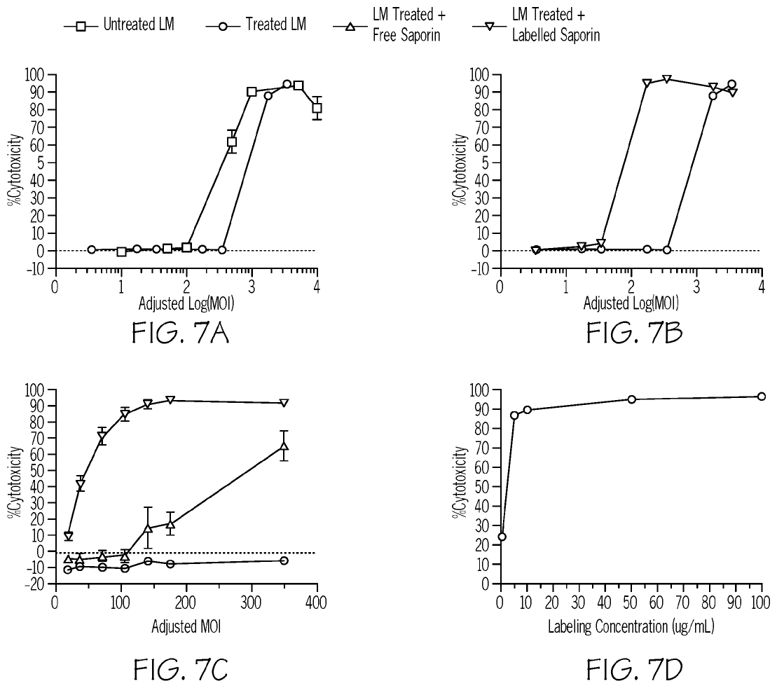

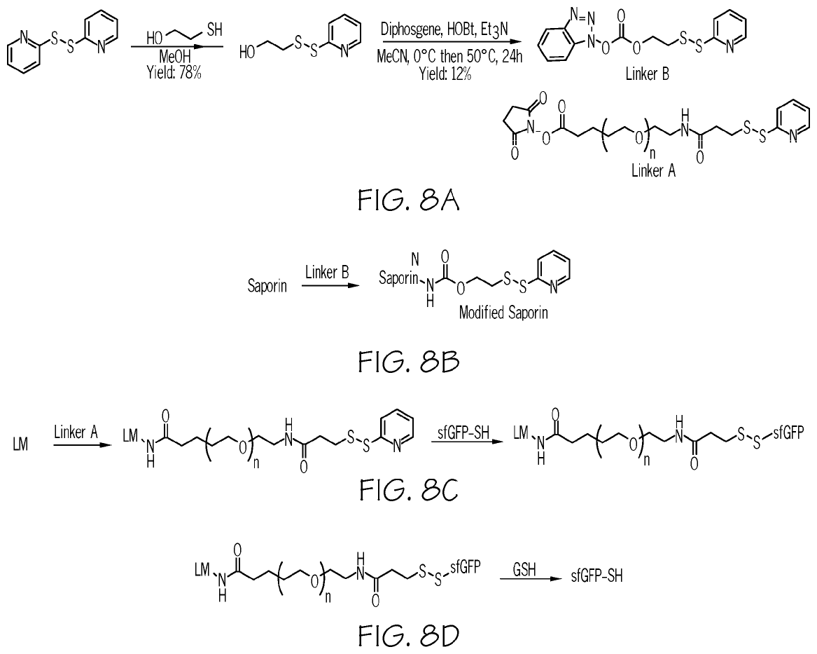

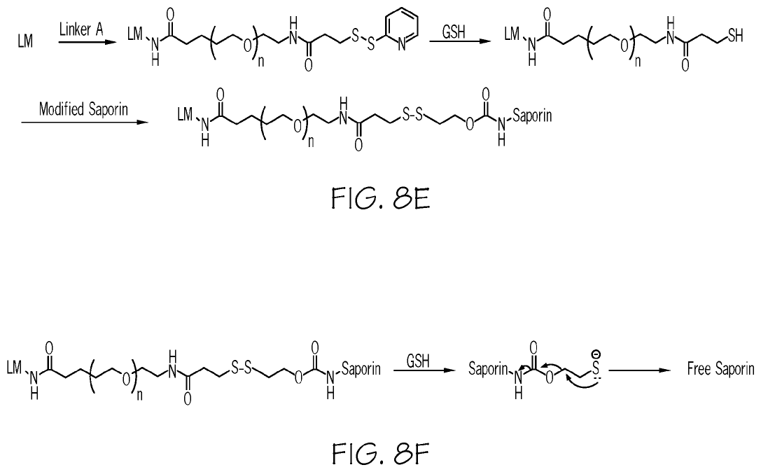

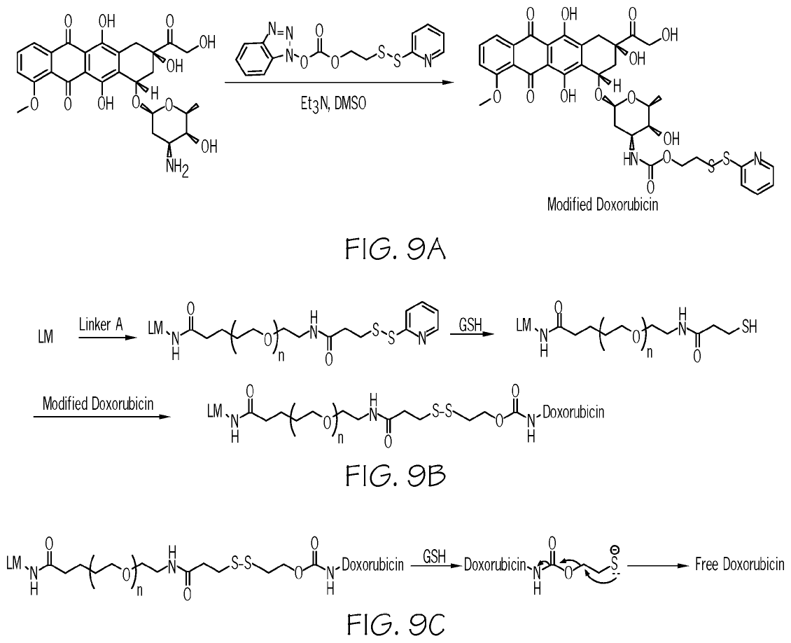

Fluorescence microscopy confirmed that sfGFP was successfully labeled onto the surface of LM LLO-Ova (FIG. 6A). After treatment with GSH, the sfGFP was cleaved off from the cell surface with more than 1,500 sfGFP molecules released from each bacterial cell (FIGS. 6B and 6C).

Fluorescence microscopy of J774 cells infected with sfGFP-LM demonstrated that sfGFP was released from the bacterial cells after entering J774 cells, bacteria cells that did not gain entry had not released their fluorescent payload (FIG. 6D). Together, these results confirmed that protein payloads which are covalently attached to the bacterial surface via a disulfide bond can be released under intracellular microenvironments and are stable in the absence of reducing agents.

To confirm successful labeling of saporin, saporin-LM was incubated with saporin antibody and subsequently a fluorescent secondary antibody for microscopy visualization. Fluorescence imaging of the saporin-LM confirmed the attachment of saporin to the bacterial surface after treatment with modified saporin.

### Example 1.6. LM Mediated Delivery of Saporin

The impact of surface functionalization for saporin loading was evaluated using a GPA SRB assay and demonstrated a loss of LM LLO-Ova intrinsic cytotoxicity by roughly fivefold (FIG. 7A). This was possibly caused by bacterial cell death due to toxicity of linker A and/or the prolonged multi-step labeling process during which the bacteria are deprived from culture media.

Moreover, saporin labeling involves direct covalent attachment of linker A to a bacterial surface protein, which can potentially compromise surface protein function and invasion. In a GPA SRB cytotoxicity assay, however, saporin labeling significantly enhances the cytotoxicity of LM LLO-Ova by approximately tenfold (FIGS. 7B and 7C). Saporin-LM also demonstrated a labeling-concentration dependent cytotoxicity against J774 (FIG. 7D).

Applicants also observed high cytotoxicity of unmodified saporin at concentrations above 1μg/mL. This is likely due to the endocytic nature of J774 cells which sample their extracellular environment. The cytotoxicity of free saporin in J774 cells demonstrates that cells actively internalize saporin molecules via pinocytic pathways.

In the case of saporin-LM, effective cell killing can scarcely be ascribed to potential presence of free saporin. LM treated with unmodified saporin causes only a marginally increase in cytotoxicity thereby indicating the majority of free saporin is washed away.

Furthermore, it is unlikely that the total saporin on labelled LM achieves the same total concentration in solution as the 1 ug/mL of free saporin. Taken together, these results demonstrate effective cytotoxic payload delivery by LM.

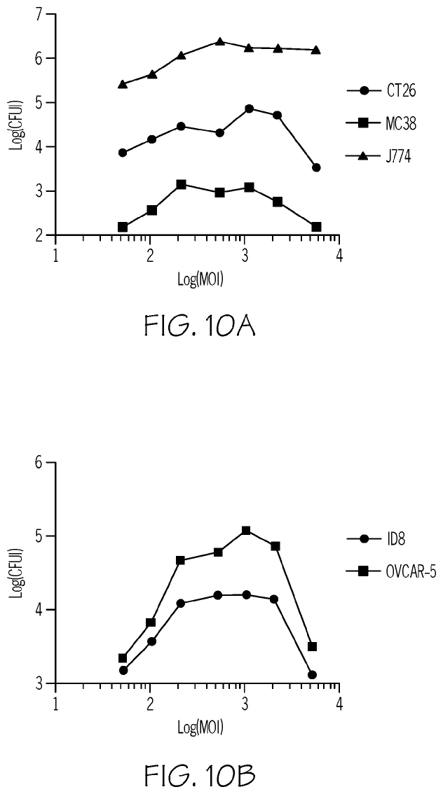

FIGS. 10A-10B illustrate differences in infectivity for LM LLO-Ova in additional cancer cell lines. FIG. 10A illustrates a comparison of infectivity between murine colorectal cancer cell lines CT26 and MC38 with J774. FIG. 10B illustrates comparison of infectivity between murine ovarian cancer ID8 cell line and human ovarian cancer cell line OVCAR-5. The data indicate that LM LLO-Ova can target different types of cancer cells for the delivery of therapeutic agents.

### Example 1.7. Discussion

In sum, Applicants demonstrate in this Example the novel utility of LM as a chemotherapeutic delivery vehicle via loading of ADC and saporin cargo on LM for delivery into cancer cells. Through experiments comparing the infectivity and cytotoxicity of multiple LM strains, it was determined the LM LLO-Ova strain had the best profile for producing efficacy in a drug delivery.

Furthermore, LM LLO-Ova strains are clinically relevant and under investigation for applications in cancer immunotherapy. LM labelled with both SN38 and Dox-ADC was able to kill cancer cells in a labelling concentration dependent manner. Interestingly, the SN38-ADC was found to be more potent than the Dox-ADC, despite a lower DAR and a reportedly higher IC50.

This difference is because of differences in the ADC linker design where the SN38-ADC is attached by a cleavable ester linker while the Dox-ADC is attached by a non-cleavable linker. Release rate directly correlates to intracellular drug concentration and therefore the improved stability offered by the non-cleavable linker likely hinders cytotoxicity.

To improve upon the ADC delivery method, Applicants also investigated covalent attachment of saporin to a chemically modified LM surface. Chemical modification of LM resulted in greater loss in cytotoxicity but enabled loading of GFP and saporin onto the cell surface. Release from LM was confirmed to be glutathione dependent as designed. Saporin delivery was found to be highly potent resulting in improved target cell killing over the ADC approach in vitro. Of the two payloads, saporin B, which is released from LM in an unmodified form, was more potent than its counterpart saporin A.

Release of unmodified protein products results in reduced steric hindrance of protein-substrate interactions. To date, many LM strains have been developed with a wide variety of genomic deletions and plasmids incorporated. When comparing the infectivity of several common LM laboratory strains, a striking similarity was observed between those strains with and without manipulation of LLO expression.

Despite a partial rescue of attenuation, the LM LLO-Ova vaccine strain infectivity closely matched that of the Δhly strain with complete deletion of LLO. LLO is reported to be highly cytotoxic to leukocytes and the major mediator of toxicity in infected endothelial cells.

Enzymatically induced membrane leakiness in cancer cells could potentially increase antigen release and tumor immunogenicity. The high similarity in infectivity of LM strains did not extend to cytotoxicity in the J774 cell line where a greater than 104-fold difference in cytotoxicity was demonstrated between the WT/4029 strains and the Δhly strain. This difference in cytotoxicity reinforces the importance of LLO in LM toxicity but also its role in the inherent anti-cancer property LM cancer vaccines.

It is likely that the cytotoxicity of the LM LLO-Ova works in conjunction with the delivered cytotoxic payloads to produce demonstrated results. Indeed, this is in part why LM is an attractive therapy vehicle because it can attack cancers through multiple mechanisms.

The data and methods presented herein establish the precedent for the continued investigation of LM as a chemotherapeutic delivery vehicle. LM based drug delivery systems are a promising alternative to modern methods of drug delivery, like nanoparticles and ADCs. Liposomes are the most widely used nano vehicle but are reported to be immunosuppressive and, therefore, pro-tumor growth.

ADCs are highly specific to target antigens but are susceptible to the development of resistance via loss or downregulation of surface targets. Both nano and ADC systems of chemotherapy delivery struggle to penetrate into tumors and, therefore, there is a need for drug delivery systems that overcome these issues facing modern drug vehicles. LM has the potential to overcome the issues facing modern drug vehicles due to its inherent properties including its immunogenicity, life cycle, and motility. Therefore, as Applicants' results demonstrate, LM is a novel chemotherapy delivery vehicle that could solve the issue associated with modern drug delivery vehicles.

In conclusion, Applicants' results demonstrate that live attenuated LM can deliver chemotherapeutic cargo into cancer cells resulting in cell killing in a payload dependent manner. This increased cytotoxicity was mediated by the delivery of an SN38/Dox-ADC and saporin payloads. This application is a departure from the reported application of LM as a radiotherapy delivery vehicle.

### Example 1.8. Listeria and J774 Cell Culture

LM were cultured in brain heart infusion (BHI) media supplemented with 34 ug/mL streptomycin at 37° C. to an OD₆₀₀ value between 0.65-0.7528,27. LM were stored at −80° C. for no longer than 2 months. LM stock density was measured using CFU counting by serial dilution and streaking on BHI agar plates (density between 1-2×10⁹ CFU/mL). The LM LLO-Ova strain was developed from the XFL-7 (ΔprfA) strain as previously described using the pGG34 plasmid which encodes a truncated Listeriolysin (LLO) fused to chicken ovalbumin (accession number NM_205152). The LM LLO-Ova strain was cultured similarly to above with the addition of 34 ug/mL chloramphenicol in BHI culture media.

The macrophage-like sarcoma cell line J774 was purchased from American Type Culture Collection (ATCC). J774 cells were maintained by culturing at 37° C. 5% CO2 in Dulbecco Modified Eagles Medium (DMEM) supplemented with 10% FBS, 4.5 g/L glucose, L-glutamine, sodium pyruvate, 10 U/mL penicillin, 10 ug/mL streptomycin, and 5 ug/mL plasmocin. Cells were tested for mycoplasma contamination before and after experiments. Subculturing of cells was performed using 0.25% trypsin supplemented with 0.54 mM EDTA and scraping.

### Example 1.9. LM Infectivity Gentamicin Protection Assay

For comparison of LM infectivity, a gentamicin protection assay was developed and adapted from reported protocols. J774 cells were passaged as normal and seeded into a 96-well tissue culture plate at a density of 32,500 cells/well. The next day, in preparation for infecting cells, LM stocks were thawed rapidly in a 37° C. water bath before being centrifuged at 5,000xg for 10 min. LM were resuspended in room temperature (RT) DMEM media supplemented with 10% FBS with no antibiotics and diluted to desired MOI densities. 22-24 hours post cell seeding J774 cells were washed once with PBS media and 100 uL of LM containing media was added to each well. The J774 cells were then placed into an incubator and cells infected for 3 hours. Following infection, the media was removed, and cells were washed once with RT PBS. 100 uL of fresh media was added and the cells lek at RT for 15 min for short recovery period followed by another 100 uL of media containing 100 ug/mL gentamicin to achieve a final gentamicin concentration of 50 ug/mL. Following another incubation period of 1 hr at 37° C. the media was removed and replaced with warm MQ H20 to lyse the cells. Following lysis, wells were thoroughly mixed by pipesng, serially diluted into PBS, and the CFU titrated on BHI agar plates.

### Example 1.10. LM Infectivity Immunofluorescence Assay

For secondary comparison of LM infectivity, J774 cells were prepared in a 96-well plate and infected with LM strains as described above. Following gentamicin treatment, instead of lysing the cells, the infected cells were washed twice with PBS and fixed with 4% paraformaldehyde for 20 min at RT. After fixation the cells were washed twice again with PBS and 100 uL of blocking and permeabilization (BP) buffer (PBS+5% BSA+0.1X Triton X-100) was added to each well. After incubating at RT for 1 hr the cells were washed three times for 5 min with PBS.

Primary antibody, rabbit anti-LM (Invitrogen cat #PA-7230), was diluted to 2 ug/mL in BP buffer and added to wells. Following overnight incubation at 4° C., cells were washed three times with PBS and labelled with anti-rabbit secondary Ab (Invitrogen cat #A11008) diluted to 1 ug/mL in BP buffer. Cells were washed again three times for 5 min with PBS and nuclei stained with DAPI for 30 min. Cells were washed again, and the total fluorescence measurements and images taken.

### Example 1.11. LM Cytotoxicity Cell Viability Assay

For comparison of LM cytotoxicity, a modified GPA assay was used in conjunction with an SRB cytotoxicity assay. J774 cells were seeded in a 96-well tissue culture plate at a density of 10,000 cells/well. The next day, LM were prepared at desired densities in antibiotic lacking media. Cells were washed once with PBS and added to the 96-well plate. Cell infection was performed at 37° C. for 3 hrs. Following infection, the media was removed, the cells washed once with PBS, and 100 uL of antibiotic lacking media added to the wells for a 15 min recovery period. 100 uL of media containing 10 ug/mL gentamicin was added to achieve a final gentamicin concentration of 5 ug/mL.

Infected cells were incubated for 48 hrs with fresh 5 ug/mL gentamicin media added after the first 24 hrs. Following the incubation period, cells were fixed with 10% TCA overnight at 4° C. Following four washes with MQ H20 the plate was air dried and 100 uL of 0.4% SRB dye in 1% acetic acid was added to each well. Cells were stained for 30 min in the dark at RT. After staining, cells were washed 4 times with 1% acetic acid solution. The plate was air dried again before adding 10 mM Tris base and absorbance at 565 nm was measured to determine cytotoxicity.

### Example 1.12. LM Cytotoxicity Annexin V PI Flow Cytometry Assay

As a secondary comparison of LM cytotoxicity, J774 cells were seeded in 6-well tissue culture plates at a density of 500,000 cells/well. The following day, LM were prepared at desired densities and infection performed as described above. After infection, the cells were washed and incubated for an additional 3 hr in 5 ug/mL gentamicin media to allow cytotoxic effects to develop while minimizing reduction of cell count.

Following incubation, cells were trypsinized and scraped from 6-well dishes to be transferred to a round bottom plate. For boiled cell controls, cells were incubated at 100° C. for 15 min. For heat-induced control cells, incubation at 60° C. for 20 min was used. Cells were washed once with blocking buffer and then incubated with anti-annexin V APC (Invitrogen cat #88-8007-72) in blocking buffer for 30 min at 37° C. in the dark.

PI stain was then immediately added, and cells incubated for 15 min at RT. Following staining, cells were washed once with blocking buffer and fixed with 2% paraformaldehyde for 30 min at 4° C. Cells were resuspended in PBS, analyzed on a BD Fortessa flow cytometer, and results quantified using FlowJo sokware version 10.7.0.

### Example 1.13. Anti-LM SN38-ADC and Dox-ADC Synthesis

The rabbit anti-LM antibody from Invitrogen (cat #PA-7230) was used for the synthesis of both SN38 and Dox ADCs. PerKit antibody conjugation kits produced by CellMosaic were used to perform antibody labeling reactions and purification of final ADC products (cat #CM11408 for SN38-ADC and CM11406 for Dox-ADC). These kits produced the anti-LM SN38-ADC with a cleavable ester linker and the anti-LM Dox-ADC with a non-cleavable linker.

The concentration of both ADCs was determined by measuring absorbance at 280 nm. The drug antibody ratio (DAR) for the SN38-ADC was determined by measuring absorbance at 280 nm and 380 nm while the Dox-ADC DAR was determined by measuring absorbance at 280 nm and 481 nm. Following measurements, the DAR of the SN38 ADC was determined to be ˜3 while the Dox-ADC DAR was determined to be ˜7. Both ADCs were freeze-dried and stored at −80°C.

### Example 1.14. LM LLO-Ova Ab/ADC Labeling

The freeze-dried ADCs were reconstituted with RT MQ water. No precipitation of SN38 or Dox-ADC was observed over the course of experimentation. LM LLO-Ova were thawed and pelleted as described above but were resuspended in PBS for labeling. LM LLO-Ova were diluted to desired densities and mixed with ADC/Ab at target concentrations. Labeling was performed at RT for 1 hr with periodic mixing. Following labeling, the LM was pelleted, and any unbound ADC and Ab removed.

For experiments evaluating the effect of LM labeling on infectivity and cytotoxicity 1×10⁹ CFU was incubated under labeling conditions. This high density of CFU minimized the loss of LM density during extended PBS incubation and subsequent resuspension. For experiments evaluating LM LLO-Ova delivery and cytotoxicity of ADC payloads LM was initially diluted to the requisite density for 100:1 MOI infection to maximize the amount of ADC/Ab per bacterium.

### Example 1.15. ADC/Ab Labelled LM LLO-Ova Infectivity and Cytotoxicity Assays

Following labeling of LM LLO-Ova with ADC/Ab, the bacteria were resuspended in antibiotic lacking media and used in GPA CFU counting infectivity and SRB cytotoxicity assays as described above without alteration. In all experiments, unlabeled LM was used for comparison and evaluating effects of payload labeling.

### Example 1.16. Synthesis of Linker B

Synthesis of 2-(2-Pyridinyldithio)ethanol: A solution of 2-Mercaptoethanol (491 μL, 7.0 mmol, 1 eq) in 10 mL methanol was added dropwise to a solution of 2,2′-dipridyl disulfide (3.00 g, 13.6 mmol, 2 eq) in 10 mL methanol. The reaction was stirred for 1.5 h under argon at room temperature. The solvent was removed under vacuum, and the residue was redissolved in 3 mL dichloromethane and purified via silica chromatography (0%-30% EtOAc/pet. Ether) to yield the desired product as a light-yellow oil (996.8 mg, 76%).

Synthesis of 1H-Benzotriazol-1-yl 2-(2-pyridinyldithio)ethyl carbonate (linker-B): To a solution of 2-(2-Pyridinyldithio)ethanol (816.6 mg, 4.36 mmol, 1 eq), HOBT (766 mg, 5.67 mmol, 1.3 eq), and triethylamine (608 μL, 4.36 mmol, 1 eq) in 15 mL acetonitrile at 0° C. was added a solution of diphosgene (350 μL, 2.92 mmol, 0.67 eq) in 5 mL acetonitrile at 0° C. The reaction mixture was stirred under argon at 0° C. for 30 minutes, and then the solution was heated to 50° C. for 36 h. The solvent was removed under vacuum and the crude product was dissolved in water with trifluoroacetic acid and purified via preparative HPLC, yielding 6 as a light-yellow solid (181.0 mg, 11.9%).

### Example 1.17. Synthesis of Modified Saporin

0.1 mg of linker-B was dissolved in 50 μL acetonitrile and 100 μL PBS and combined with 100 μL PBS containing 100μg saporin (Sigma Aldrich, S9896). The mixture was stirred at room temperature for 1 hour. After purification through a centrifugal filter (Amicon Ultra-4, molecular weight cut-off 10 kDa) the concentration of the final product was adjusted to 0.5 mg/mL.

### Example 1.18. LM LLO-Ova SfGFP Labelin,. Quantification, and Confirmation of Payload Delivery

The variant of sfGFP (PBD:2B3P) used in this Example was modified by adding cysteine residues at N and C termini. The product was expressed and purified. The sfGFP stock solution, originally in 50 mM HEPES (pH 7.4), 150 mM NaCl, 5 mM DTT at a concentration of 2 mg/mL, was desalted using Amicon Ultra-4 30 kDa centrifuge filter. To label sfGFP, 5.0×109 CFU of LM LLO-Ova was suspended in 2.5 mL PBS containing 25 mg linker-A (SPDP-PEG-Succinimidyl Valerate MW 5000, BroadPharm, BP-25336) and stirred for 15 min at 37° C.

After three washes with PBS the cells were resuspended in 0.5 mL PBS containing 50 μg desalted sfGFP and stirred for 20 min at 37° C., followed by three washes and resuspension in 1 mL PBS. To quantify labeled sfGFP on LM LLO-Ova, the sfGFP-labeled cells were treated with 200μL PBS containing 0.1 M GSH at 37° C., with treatment of 200 μL plain PBS as negative control. The cells were centrifugated, separated from the supernatant, and resuspended in 100 μL PBS. Cell suspensions and supernatants were analyzed via spectrofluorometry at excitation/emission wavelengths of 475/509 nm. To confirm intracellular delivery of sfGFP, 2.5×106 J774 cells were seeded in a Corning 25 cm² cell culture flask for 22-24 hours. The following day the J774 cells were infected with 5.0×109 units (2,000 MOI) of sfGFPlabeled LM LLO-Ova cells for 1 hour at 37° C. with 5% CO₂. The J774 cells were detached from the culture flask by trypsin, fixed by 4% formaldehyde solution, and analyzed via fluorescence microscopy.

### Example 1.19. LM LLO-Ova Saporin Labeling and Confirmation of Labeling

To label LM with saporin, 1.0×10⁹ CFU of LM LLO-Ova was suspended in 0.5 mL PBS containing 5 mg linker A and stirred for 15 min at 37° C. After removal of PBS the cells were resuspended in 1 mL PBS containing 100 mM GSH and stirred for 15 min at 37° C. After three washes with 1 mL PBS the cells were resuspended 0.1 mL PBS containing 10 μg modified saporin and stirred for 20 min at 37° C., followed by three washes and resuspension in 1 mL PBS. To confirm saporin labeling, the labeled cells were resuspended in 0.5 mL PBS containing 5 μg/mL rabbit saporin polyclonal antibody (ThermoFisher, PA1-18425) and stirred for 15 min at 37° C.

After three washes with 1 mL PBS the cells were resuspended 0.5 mL PBS containing 4 μg/mL goat anti-rabbit secondary antibody (ThermoFisher, A-11008), followed by three washes and resuspension in 1 mL PBS. Cell suspensions were analyzed via fluorescence microscopy to compare relative fluorescence intensity.

### Example 1.20. Saporin Labeled LM LLO-Ova Cytotoxicity Assays

Following labeling of LM LLO-Ova with saporin the bacteria were resuspended in antibiotic lacking media and used in SRB cytotoxicity assays as described above without alteration. In all experiments untreated LM and LM treated with only linker A were used as controls to evaluate effects of payload labeling on cytotoxicity.

### Example 1.21. Fluorescent Microscopy Sample Preparation, Image Acquisition, and Image Processing

1% agarose pads (2 mm thick) were prepared, and 8×8 mm square pads were cut out for microscopy visualization of bacteria or sacculi samples. 1 μL of each sample was spo Yed on a 22×60 mm coverslip and covered with an 1% agarose pad. The coverslip was then placed in a slide holder on the microscope with the pad facing upwards. Phase contrast images and fluorescence images were acquired using Nikon Ti-e inverted microscope equipped with a 1.4 NA Plan Apo 100x oil objective and Andor iXon X3 EMCCD camera. NIS-Elements AR sokware was used for image acquisition. The fluorescence images were obtained using Lumencor Spectra X engine with excitation filter 470/40 nm (FTIC) and emission filter 525/50 nm (FTIC). All images were processed via FIJI sokware and a MicrobeJ plugin, allowing identification of bacterial cells and fluorescence measurements.

### Example 1.22. Statistical Analysis

All statistical analysis was performed using GraphPad Prism 10 version 10.0.0. For comparison of LM infectivity immunofluorescence measurements, a one-way ANOVA with multiple comparison was performed. Comparison of LM infection cytotoxicity by flow cytometry used a one-way ANOVA with multiple comparison as well. Comparison of consistency between LM stock preparations and methods of preparation was performed using an F-test. Statistical comparison of ADC delivery cytotoxicity was performed using an unpaired student t-test. Significant p values for all comparisons are illustrated in figures in the following way: *p value<0.05, **p value<0.01, ***p value<0.001, and ****p value<0.0001.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A modified Listeria monocytogenes bacterium (Listeria), wherein the Listeria comprises at least one therapeutic agent associated with the Listeria.

2. The Listeria of claim 1, wherein the therapeutic agent is associated with the surface of the Listeria.

3. The Listeria of claim 2, wherein the therapeutic agent is associated with the surface of the Listeria through a Listeria binding agent, wherein the Listeria binding agent is covalently associated with the therapeutic agent and non-covalently associated with the surface of the Listeria.

4. The Listeria of claim 3, wherein the Listeria binding agent comprises an antibody or a fragment thereof.

5. The Listeria of claim 3, wherein the therapeutic agent is covalently associated with the Listeria binding agent through a cleavable linker.

6. The Listeria of claim 3, wherein the therapeutic agent is covalently associated with the Listeria binding agent through a non-cleavable linker.

7. The Listeria of claim 3, wherein the Listeria comprises a plurality of different therapeutic agents associated with a plurality of Listeria binding agents.

8. The Listeria of claim 3, wherein the Listeria comprises at least a first therapeutic agent associated with a first Listeria binding agent, and a second therapeutic agent associated with a second Listeria binding agent.

9. The Listeria of claim 2, wherein the therapeutic agent is covalently associated with the surface of the Listeria through a cleavable linker, wherein the cleavable linker directly links the therapeutic agent to the surface of the Listeria.

10. The Listeria of claim 2, wherein the therapeutic agent is covalently associated with the surface of the Listeria through a non-cleavable linker, wherein the non-cleavable linker directly links the therapeutic agent to the surface of the Listeria.

11. The Listeria of claim 1, wherein the therapeutic agent is selected from the group consisting of an anti-cancer agent, a chemotherapeutic agent, non-radioactive compounds, cytotoxic proteins, Doxorubicin, Saporin, SN38, derivatives thereof, and combinations thereof.

12-14. (canceled)

15. The Listeria of claim 1, wherein the Listeria is attenuated.

16. The Listeria of claim 15, wherein the attenuated Listeria comprises a mutation or deletion in one or more virulence genes selected from the group consisting of prfA. actA, hly, and combinations thereof.

17-19. (canceled)

20. A method of delivering at least one therapeutic agent to a subject, said method comprising:
administering to the subject a modified Listeria monocytogenes bacterium (Listeria), wherein the Listeria is associated with the therapeutic agent.

21. The method of claim 20, wherein the administering comprises systemic administration to a specific tissue of a subject.

22. The method of claim 21, wherein the tissue comprises a tumor.

23. The method of claim 20, wherein the method is utilized to treat or prevent a condition in the subject.

24. The method of claim 23, wherein the condition is cancer.

25. The method of claim 24, wherein the cancer is selected from the group consisting of ovarian cancer, colorectal cancer, sarcoma, hepatocellular carcinoma, and combinations thereof.

26. The method of claim 20, wherein the Listeria is administered to the subject in combination with one or more additional treatments.

27. The method of claim 26, wherein the one or more additional treatments is selected from the group consisting of anti-cancer vaccines, chemotherapy, radiotherapy, immunotherapy, or combinations thereof.

28. The method of claim 20, wherein the subject is a human being.

29. The method of claim 20, wherein the therapeutic agent is associated with the surface of the Listeria.

30. The method of claim 29, wherein the therapeutic agent is associated with the surface of the Listeria through a Listeria binding agent, wherein the Listeria binding agent is covalently associated with the therapeutic agent and non-covalently associated with the surface of the Listeria.

31. The method of claim 30, wherein the Listeria binding agent comprises an antibody or a fragment thereof.

32. The method of claim 30, wherein the therapeutic agent is covalently associated with the Listeria binding agent through a cleavable linker.

33. The method of claim 30, wherein the therapeutic agent is covalently associated with the Listeria binding agent through a non-cleavable linker.

34. The method of claim 30, wherein the Listeria comprises a plurality of different therapeutic agents associated with a plurality of Listeria binding agents.

35. The method of claim 30, wherein the Listeria comprises at least a first therapeutic agent associated with a first Listeria binding agent, and a second therapeutic agent associated with a second Listeria binding agent.

36. The method of claim 29, wherein the therapeutic agent is covalently associated with the surface of the Listeria through a cleavable linker, wherein the cleavable linker directly links the therapeutic agent to the surface of the Listeria.

37. The method of claim 29, wherein the therapeutic agent is covalently associated with the surface of the Listeria through a non-cleavable linker, wherein the non-cleavable linker directly links the therapeutic agent to the surface of the Listeria.

38. The method of claim 20, wherein the therapeutic agent is selected from the group consisting of an anti-cancer agent, a chemotherapeutic agent, non-radioactive compounds, cytotoxic proteins, Doxorubicin, Saporin, SN38, derivatives thereof, and combinations thereof.

39-41. (canceled)

42. The method of claim 20, wherein the Listeria is attenuated.

43. The method of claim 42, wherein the attenuated Listeria comprises a mutation or deletion in one or more virulence genes selected from the group consisting of prfA, actA, hly, and combinations thereof.

44. (canceled)

45. A method of making a modified Listeria monocytogenes bacterium (Listeria), said method comprising associating the Listeria with at least one therapeutic agent.

46-61. (canceled)
